(19) United States
(12) Patent Application Publication
(10) Pub. No.: US 2007/0179328 A1
(43) Pub. Date: Aug. 2, 2007

(54) IMPACT TOOL

(76) Inventors: Takuhiro Murakami, Ibaraki (JP); Junichi Kamimura, Ibaraki (JP); Katsuhiro Oomori, Ibaraki (JP); Shinki Ohtsu, Ibaraki (JP); Hiroto Inagawa, Ibaraki (JP)

Correspondence Address: MATTINGLY, STANGER, MALUR & BRUNDIDGE, P.C., 1800 DIAGONAL ROAD, SUITE 370, ALEXANDRIA, VA, 22314, US

(21) Appl. No.: 11/627,574

(22) Filed: Jan. 26, 2007

(30) Foreign Application Priority Data
Feb. 1, 2006 (JP) P2006-024266

(51) Int. Cl. C07C 2/64

(52) U.S. Cl. 585/448

(57) ABSTRACT

An impact tool, which can realize the reduction of noise without inviting the reduction of a fastening ability and which can improve the durability of a damper while preventing its damage. The impact tool includes a rotary impact mechanism mounted on a spindle to be rotationally driven by a motor, so that rotary impact is applied to a tip tool by transmitting the rotary impact intermittently from a hammer through an anvil to the tip tool. A plurality of pawls are formed on two half members of the anvil in the axial direction. A rubber damper is disposed in a space between the pawls arranged alternately in the circumferential direction of the two half members. The minimum sectional area of the space formed between the pawls is set larger than the sectional area of the rubber damper.

### BACKGROUND OF THE INVENTION

The present invention relates to an impact tool for generating a rotary impact to perform desired works such as a screw fastening operation.

The impact tool as one mode of the electric tool performs a screwing operation by generating a rotary impact with a motor as a drive source to rotate a tip tool and by applying the impact intermittently to the tip tool. The impact tool is widely used at present because it is advantageous in a small reaction and in a high fastening ability. However, the impact tool has a rotary impact mechanism for generating the rotary impact, so that it has such a serious noise as raises problems.

FIG. 10 is a longitudinal section showing a general impact tool used in the prior art.

The impact tool of the prior art shown in FIG. 10 is enabled to transmit the rotary impact intermittently to a tip tool 4 thereby to perform the screwing operation, by using a battery pack 1 as an electric source and a motor 2 as the driving source to drive a rotary impact mechanism unit thereby to apply the rotations and impact to an anvil 3.

In the rotary impact mechanism unit mounted in a hammer case 5 of this impact tool, the rotation of the output shaft (or the motor shaft) of the motor 2 is decelerated through a planetary gear mechanism 6 and transmitted to a spindle 7, so that the spindle 7 is rotationally driven at a predetermined speed. Here, the spindle 7 and a hammer 8 are connected through a cam mechanism, which is constituted to include a V-shaped spindle cam groove 7a formed in the outer circumference of the spindle 7, a V-shaped hammer cam groove 8a formed in the inner circumference of the hammer 6, and balls 9 engaging with those cam grooves 7a and 8a.

Moreover, the hammer 8 is urged at all times in the direction toward the tip by a spring 10, and is so positioned at a still time by the engagement between the balls 9 and the cam grooves 7a and 8a as is spaced from the end face of the anvil 3. Moreover, protrusions are individually symmetrically formed at the two portions on the confronting rotary faces of the hammer 8 and the anvil 3. Here, a screw 11, the tip tool 4 and the anvil 3 are restricted in the rotating directions from one another. In FIG. 10, moreover, numeral 14 designates a bearing metal for bearing the anvil 3 rotatably.

The rotation of the spindle 7 is transmitted, when the spindle 7 is rotationally driven, through the aforementioned cam mechanism to the hammer 8, and the protrusion of the hammer 8 come, before the hammer 8 makes a half rotation, into engagement with the protrusion of the anvil 3. If relative rotations are caused between the hammer 8 and the spindle 7 by the engagement reaction at that time, the hammer 8 begins to come back to the motor 2 while compressing the spring 10 along the spindle cam groove 7a of the cam mechanism.

When the protrusion of the hammer 8 rises, as the hammer 8 retracts, over the protrusion of the anvil 3 thereby to release their engagement, the hammer 8 is moved forward by the urging force of the spring 10 while being abruptly accelerated rotationally and forward by the elastic energy stored in the spring 10 and the action of the cam mechanism in addition to the rotating force of the spindle 7, so that the protrusion restores its engagement with the protrusion thereby to begin the integral rotation. At this time, the strong rotary impact is applied to the anvil 3 so that the rotary impact is transmitted to the screw 11 through the tip tool mounted on the anvil 3.

From now on, similar actions are repeated to transmit the rotary impact is intermittently repeated and transmitted from the tip tool 4 to the screw 11 so that the screw 11 is driven into timber 12 or the fastening object.

Here, during the work using that impact tool, the hammer 8 performs the rotational motions and the longitudinal motions at the same time, so that those motions act as vibration sources to vibrate the timber 12 or the fastening object in the axial directions through the anvil 3, the tip tool 4 and the screw 11 thereby to generate a large noise.

Here, it has been found that the noise energy from the fastening object takes a large ratio in the noise at the working time using the impact tool. For reducing the noise, it is necessary to suppress the vibrating force to be transmitted to the fastening object. For this necessity, various counter-measures have been investigated (as referred to Patent Documents 1 and 2, for example).

[Patent Document 1] JP-A-7-237152

[Patent Document 2] JP-A-2002-254335

In the description of Patent Document 1, the axial force to act on the tip tool or the screw is decreased to reduce the noise by dividing the anvil into two members to form a torque transmission unit between the two members and by fitting a shock absorbing member in an axial gap. Here, a rectangular recess is formed in one of the two members, and a rectangular protrusion is formed in the other, so that the torque transmission unit is constituted to have rectangular uneven shapes and splined shapes for connecting the two members irrotationally.

When the torque is applied to the torque transmission unit, however, a high frictional force is established between the two members thereby to obstruct the relative movement of the two members in the axial direction. As a result, the axial force to act on the tip tool or the screw cannot be reduced so much as to make the noise reducing effect insufficient.

In the description of Patent Document 2, on the other hand, the torque transmission unit is constituted by using rolling parts such as balls or rollers as key elements and by bringing the grooves formed in the two halved members of the anvil and those key elements, so that the frictional force in the two members in the axial direction is reduced.

With this constitution, however, the facial pressure on the contact portions between the key elements and the grooves is so high as to raise the problems that the parts are prematurely worn, and that the structure is complicated to raise the manufacturing cost.

### SUMMARY OF THE INVENTION

The invention has been conceived in view of the problems thus far described, and has an object to provide an impact tool, which can realize the reduction of noise without inviting the reduction of a fastening ability.

The invention has another object to provide an impact tool, which can improve the durability of a damper while preventing its damage.

In order to achieve the aforementioned objects, according to the invention, there is provided an impact tool including a rotary impact mechanism mounted on a spindle to be rotationally driven by a motor, so that a rotary impact generated by the rotary impact mechanism is applied to a tip tool by transmitting the rotary impact intermittently from a hammer through an anvil to the tip tool. The impact tool includes a plurality of pawls that are formed on the axially confronting faces of two half members formed by halving the anvil in the axial direction; a damper disposed in a space formed between the pawls arranged alternately in the circumferential direction of the two half members; and the minimum sectional area S1 of the space formed between the pawls is set larger than the sectional area S2 of the damper.

In the invention the damper has a plurality of damper members of an elliptical column shape arranged in the circumferential direction around a ring-shaped connecting portion and formed integrally; and each of the damper members is arranged in the space formed between the pawls of the two half members of the anvil, so that its longer axis is directed in the circumferential direction whereas its shorter axis is arranged in the radial direction.

Further in the invention the longer axis length x of the damper members is set equal to the enveloping circle diameter d of the space formed between the pawls of the two half members of the anvil; and the shorter axis length y of the damper members is set smaller than the enveloping circle diameter d.

Still further in the invention of the shorter axis length y of the damper members is set larger than the maximum value δ2max of the inter-pawl gap between the two half members of the anvil.

According to the invention, the damper is interposed between the two half members formed by halving the anvil in the axial direction. As a result, the vibration from the rotary impact mechanism or the vibration source is absorbed to suppress the propagation of the vibration to the fastening object thereby to reduce the noise of the impact tool.

According to the invention, moreover, the transmission torque of the anvil is increased to enlarge the relative rotations of the two half members of the anvil. Even if the space formed between the pawls becomes small, the minimum sectional area S1 thereof is set larger than the sectional area S2 of the damper arranged in that space. As a result, the elastic deformation of the damper is reduced to prevent the damage of the damper thereby to improve the durability of the same.

Still further according to the invention, each of the damper members is so arranged in the space formed between the pawls of the two half members of the anvil that its longer axis is directed in the circumferential direction whereas its shorter axis is directed in the radial direction. According to the invention of claim 3, the longer axis length x of each of the damper members is set equal to the enveloping circle diameter d of the space formed between the pawls of the two half members of the anvil, and the shorter axis length y of the same damper members is set smaller than the enveloping circle diameter d. As a result, the sectional area S2 of the damper can be set smaller than the minimum sectional area S1 of the space between the pawls of the anvil, and the damper can be assembled without any looseness between the half members.

Still further yet according to the invention, moreover, the shorter axis length y of the damper members of the damper is set larger than the maximum value δ2max of the inter-pawl gap between the two half members of the anvil. Even if the damper member should be disconnected by a cracking or the like from the connecting portion, the damper member disconnected is not flown away from the anvil by a centrifugal force, but the shock absorbing action by the damper can be stably performed.

### BRIEF DESCRIPTION OF THE DRAWINGS

FIG. 1 is a longitudinal section showing a rotary impact mechanism unit of an impact tool according to the invention.

FIG. 2 is a detailed diagram showing a portion A in FIG. 1 in an enlarged scale.

FIG. 3 is an exploded perspective view showing the rotary impact mechanism unit of the impact tool according to the invention.

FIG. 4 is an exploded perspective view showing the rotary impact mechanism unit of the impact tool according to the invention.

FIG. 5 is a sectional side elevation of an anvil of the impact tool according to the invention.

FIG. 6 is a sectional view taken along line B-B of FIG. 5.

FIG. 7 is an enlarged sectional view taken along line C-C of FIG. 6.

FIG. 8(a) is a front elevation of rubber damper, and FIG. 8(b) is a side elevation of the same rubber damper.

FIGS. 9(a) and 9(b) are front elevations for explaining the behaviors of anvil pawls.

FIG. 10 is a longitudinal section of the impact tool of the prior art.

### DETAILED DESCRIPTION OF THE INVENTION

An embodiment of the invention is described in the following with reference to the accompanying drawings.

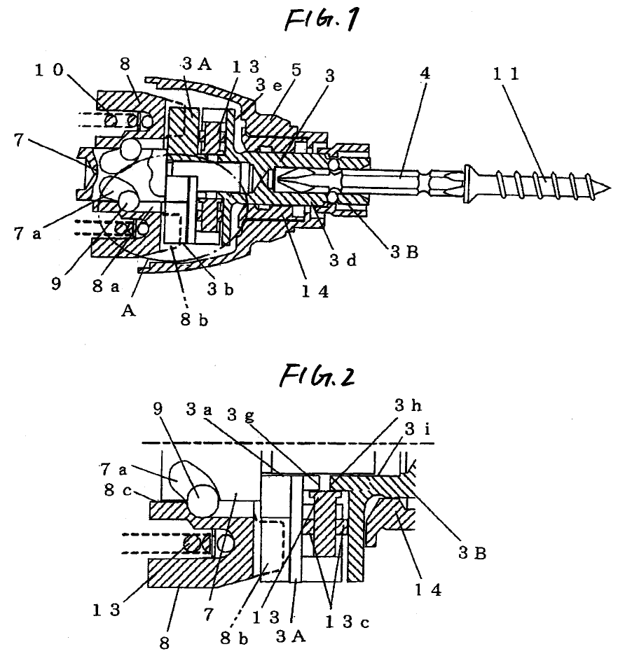

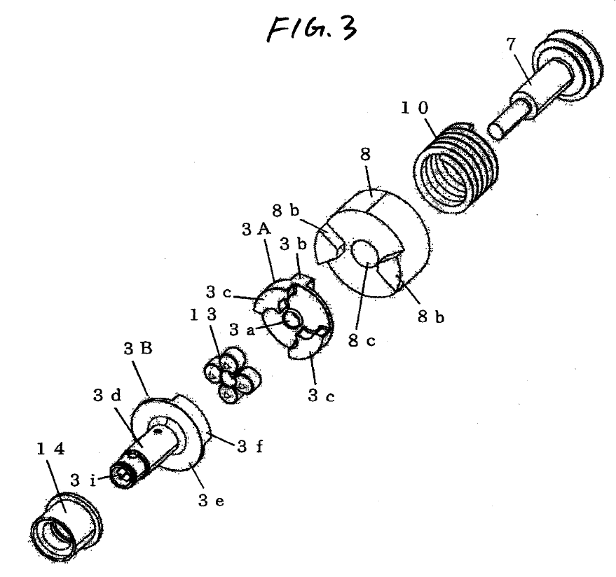

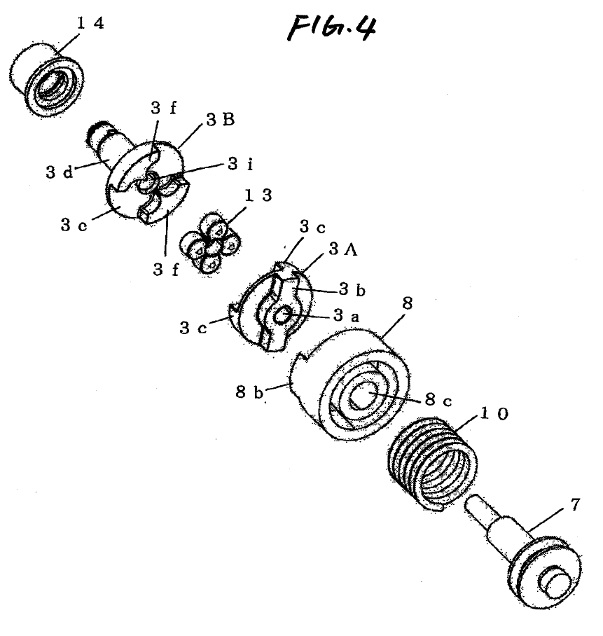

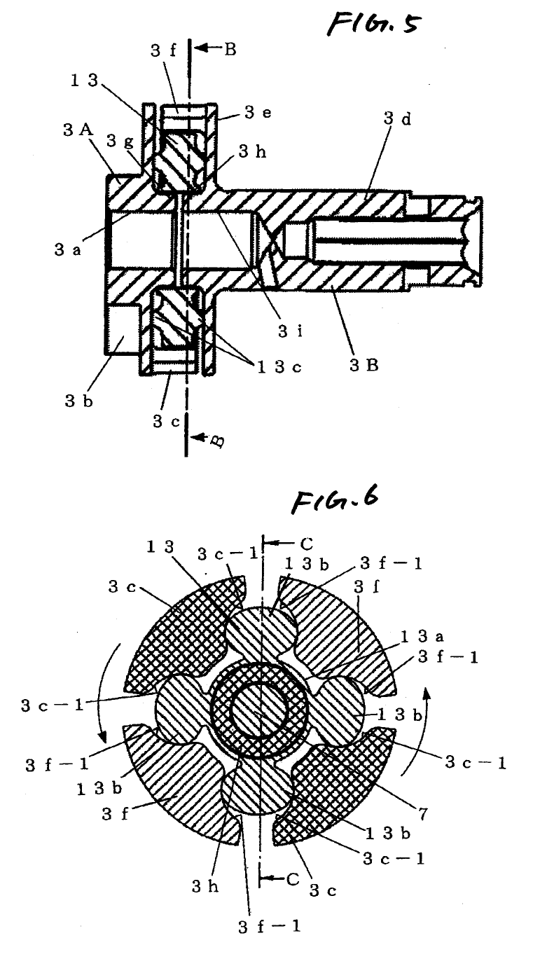

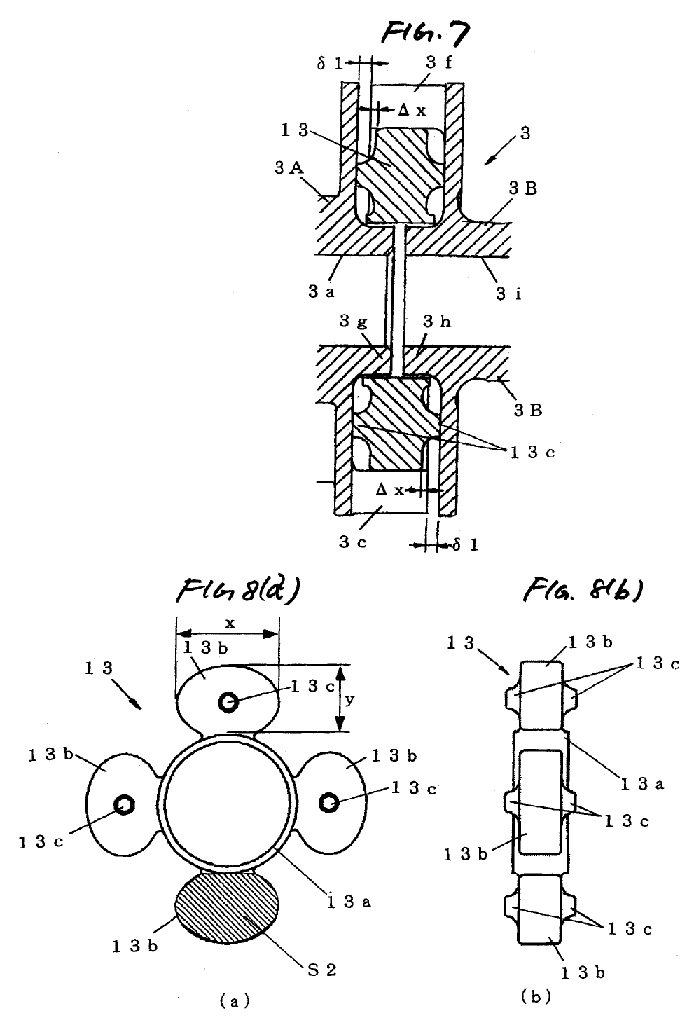

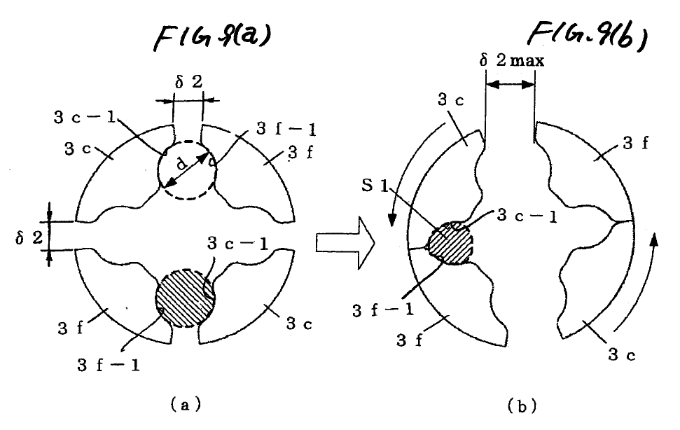

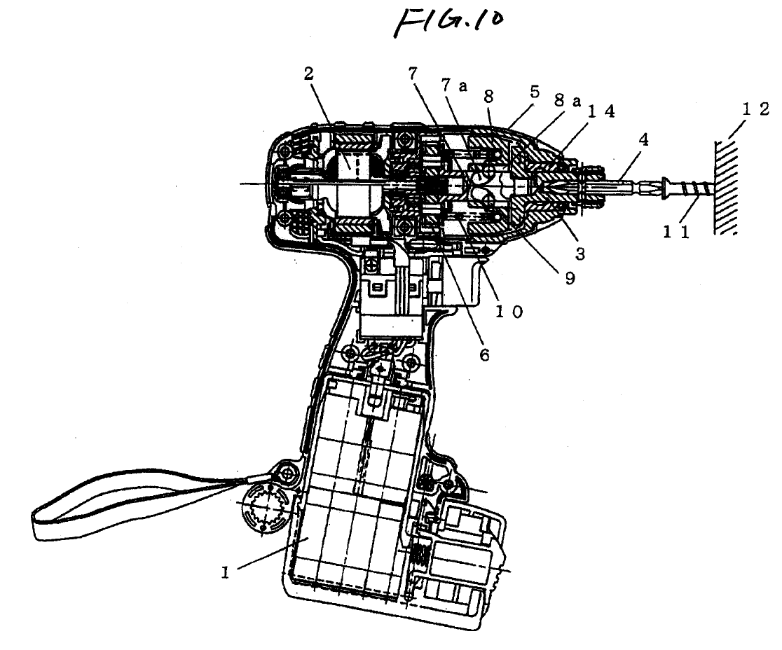

FIG. 1 is a longitudinal section of a rotary impact mechanism unit of an impact tool according to the invention; FIG. 2 is an enlarged detailed diagram of a portion A of FIG. 1; FIG. 3 and FIG. 4 are exploded perspective views of the rotary impact mechanism of the same impact tool; FIG. 5 is a sectional side elevation of an anvil of the same impact tool; FIG. 6 is a sectional view taken along line B-B of FIG. 5; FIG. 7 is a sectional view taken along line C-C of FIG. 6; FIG. 8(a) is a front elevation of a rubber damper; FIG. 8(b) is a side elevation of the same rubber damper; and FIGS. 9(a) and 9(b) are front elevations for explaining the behaviors of pawls of the anvil. In these Figures, the same elements as those shown in FIG. 10 are designated by the common reference numerals.

The impact tool according to this embodiment is a cordless hand-holdable tool having a motor as a drive source, and is similar in its constitution except a portion to that of the conventional impact tool shown in FIG. 10. Therefore, the following description is made exclusively on the featuring constitution of the invention, while omitting the repeated description on the same constitution as that shown in FIG. 10.

The impact tool according to this embodiment is characterized in that an anvil 3 is equipped with a shock absorbing mechanism. Here, the shock absorbing mechanism performs a shock absorbing function in the rotating direction and in the axial direction, and transmits a preset or higher level of torque directly. Specifically, the anvil 3 is constituted to include axially halved split members 3A and 3B, between which a rubber damper 13 is interposed as a shock absorber.

One half member 3A is molded in a generally circular shape, and has a circular hole 3a formed at its center (as referred to FIG. 3 and FIG. 4). Moreover, the half member 3A is integrally provided, on the side of a hammer 8, with a straight protrusion 3b extending through the center, as shown in FIG. 4. The hammer 8 is integrally provided, on its one end face (or on the end face confronting the half member 3A), with two sector-shaped protrusions 8b which are formed at symmetric positions spaced at 180 degrees in the circumferential direction, as shown in FIG. 3. These protrusions 8b and the aforementioned protrusion 3b formed on the half member 3A intermittently come into engagement/disengagement at every half rotations, as will be described hereinafter.

The half member 3A is further integrally provided, on the other end face (or on the end face confronting the other half member 3B), as shown in FIG. 3, with two pawls 3c which are formed at symmetric positions spaced at 180 angles in the circumferential direction. Each pawl 3c is provided with two arcuate recesses 3c-1, as shown in FIG. 6. As shown in FIG. 3 and FIG. 4, a circular hole 8c is formed through the center portion of the hammer 8.

The other half member 3B is constituted, as shown in FIG. 3 and FIG. 4, by forming a disc-shaped flange portion 3e integrally with one end portion of a hollow stem 3d and in a direction perpendicular to the axis. The other half member 3B is integrally provided, on the other end face of the flange portion 3e (or on the end face confronting the half member 3A), as shown in FIG. 4, with two pawls 3f which are formed at symmetric positions spaced at 180 angles in the circumferential direction. Each pawl 3f is provided with two arcuate recesses 3f-1.

On the other hand, the rubber damper 13 is constituted, as shown in FIG. 6 and FIG. 8, by arraying and integrating four damper members 13b having an elliptical column shape circumferentially ad at an equal angle pitch (of 90 degrees) around a ring-shaped center connecting portion 13a. As shown in FIG. 7 and FIG. 8, moreover, column-shaped protrusions 13c are protruded perpendicularly and integrally from the central portions of the two faces of each of the damper members 13b of the rubber damper 13.

Thus, the rubber damper 13 is sandwiched between the half members 3A and 3B of the anvil 3, as shown in FIG. 1 to FIG. 7. As detailed in FIG. 7, moreover, sleeve-shaped protrusions 3g and 3h are integrally protruded in the axial direction the confronting radially inner portions of the half members 3A and 3B of the anvil 3. The ring-shaped connecting portion 13a of the rubber damper 13 is fitted on the outer circumferences of those protrusions 3g and 3h. Specifically, the rubber damper 13 is arranged on the outer circumference sides of the protrusions 3g and 3h protruded from the radially inner portions of the half members 3A and 3B of the anvil 3 so that the radially inner portions are protected against the direct contact with a spindle 7 by the protrusions 3g and 3h of the anvil 3. Incidentally, a circular hole 3i is formed in the axially center portion of the half member 3B of the anvil 3.

As shown in FIG. 6, moreover, the two pawls 3c and 3f, which are formed on the confronting end faces of the half members 3A and 3B of the anvil 3, are arranged alternately of the circumferential direction, and the individual damper members 13b of the rubber damper 13 are arranged in the spaces which are formed between the individual recesses 3c-1 and 3f-1 of the pawls 3c and the pawls 3f adjoining in the circumferential direction. Here in the spaces which are formed between the circumferentially adjoining pawls 3c and 3f of the half members 3A and 3B of the anvil 3, the damper members 13b of the rubber damper 13 are so circumferentially arranged that their longer axis sides are clamped between the pawls 3c and 3f, and are so arranged that their shorter axis sides are radially directed.

Here, FIG. 9(a) shows the arrangement, in which the pawls 3c and 3f formed at the half members 3A and 3B of the anvil 3 are not loaded. In this arrangement, a diameter d of the enveloping circle of the space, which is formed by the recesses 3c-1 and 3f-1 formed in the circumferentially adjoining pawls 3c and 3f, and a longer axis length x (as referred to FIG. 8(a)) of each of the damper members 13b of the rubber damper 13 are set equal (i.e., d=x), and the shorter axis length y (as referred to FIG. 8(a)) of each of the damper members 13b of the rubber damper 13 is set smaller than the diameter d of the enveloping circuit (i.e., y<d), as shown in FIG. 9(a).

As detailed in FIG. 7, moreover, the rubber damper 13 axially abuts against the half members 3A and 3B of the anvil 3 through the protrusions 13 axially protruding from the two faces of its rubber damper 13. In the no-load state, in which the rotary impact does not act on the anvil 3, an axial gap δ1 is formed, as shown, between the pawl 3c of the half member 3A of the anvil 3 and the end face of the flange portion 3e of the other half member 3B. Likewise, the axial gap δ1 is also formed, as shown, between the pawl 3f of the other half member 3B of the anvil 3 and the end face of the half member 3A.

As detailed in FIG. 7, on the other hand, one end face (or the right end face in FIG. 7) of each of the damper members 13b of the rubber damper 13 is positioned on the axially inner side (or the left side of FIG. 7) by Δx, as shown, than the end face of the pawl 3c of the half member 3A of the anvil 3. Likewise, the other end face (or the left end face in FIG. 7) of each of the damper members 13b of the rubber damper 13 is positioned on the axially inner side (or the right side of FIG. 7) by Δx, as shown, than the end face of the pawl 3f of the other half member 3B of the anvil 3.

As shown in FIG. 1, the anvil 3 is so housed in a hammer case 5 that the stem 3d of the half member 3B is rotatably borne by a bearing metal 14. The other half member 3A is so assembled with the end face of the flange portion 3e of the half member 3B that their pawls 3c and 3f are arrayed alternately in the circumferential direction, as shown in FIG. 6. The half member 3A is supported (as referred to FIG. 2) rotatably and axially movably relative to the half member 3B by the leading end portion of the spindle 7 inserted into the circular hole 3i formed at the center thereof. As shown in FIG. 2, the spindle 7 has its leading end portion fitted in the circular hole 3i of the other half member 3B through the circular hole 3a of the half member 3A of the anvil 3.

Here in the state where the anvil 3 is housed in the hammer case 5, as described above, a space contouring the rubber damper 13 is formed by the recesses 3c-1 and 3f-1 which are formed by the pawls 3c and 3f arranged alternately in the circumferential direction of the two half members 3A and 3B. The rubber damper 13 is fitted and housed in that space, as shown in FIG. 6.

Thus in the no-load state where the rotary impact does not act on the anvil 3, a circumferential gap δ2 is formed between the pawls 3c and 3f of the two half members 3A and 3B, as shown in FIG. 6 and FIG. 9(a), and the axial gap 61 (as referred to FIG. 7) is formed between the two half members 3A and 3B, as described hereinbefore. At the time of no load, therefore, the half members 3A and 3B of the anvil 3 makes no direct contact circumferentially or longitudinally.

A tip tool 4 is removably mounted in the stem 3d of the half member 3B of the anvil 3, and the hammer 8, which is equipped with the protrusion 7b to be brought into and out of engagement with the protrusion 3b formed at the outer end face of the half member 3A, is urged at all times to the anvil 3 (or toward the leading end) by a spring 10.

Next, the description is made on the actions of the impact tool thus constituted.

In the rotary impact mechanism unit, the rotations of the output shaft (or the motor shaft) of the motor are reduced in speed through a planetary gear mechanism and transmitted to the spindle 7 so that the spindle 7 is rotationally driven at a predetermined speed. When the spindle 7 is thus rotationally driven, its rotations are transmitted through a cam mechanism to the hammer 8, so that the protrusion 8b comes, before the hammer 8 makes a half rotation, into engagement with the protrusion 3b of the half member 3A of the anvil 3 thereby to rotate the half member 3A.

When the hammer 8 and the spindle 7 are rotated relative to each other by the reaction (or the engagement reaction) accompanying the engagement between the protrusion 8b of the hammer 8 and the protrusion 3b of the half member 3A of the anvil 3, the hammer 8 starts its backward movement toward the motor along a spindle cam groove 7a of the cam mechanism while compressing the spring 10.

When the protrusion 8b of the hammer 8 rises, as the hammer 8 retracts, over the protrusion 3b of the half member 3A of the anvil 3 thereby to release their engagement, the hammer 8 is moved forward by the urging force of the spring 10 while being abruptly accelerated rotationally and forward by the elastic energy stored in the spring and the action of the cam mechanism in addition to the rotating force of the spindle 7, so that the protrusion 8b restores its engagement with the protrusion 3b thereby to begin the rotation of the anvil 3. At this time, the strong rotary impact is applied to the anvil 3. This anvil 3 is constituted by interposing the rubber damper 13 between the two half members 3A and 3B. The axial gap δ1 is formed between the two half members 3A and 3B, as shown in FIG. 7, the impact vibrations are absorbed and attenuated by the elastic deformation of the rubber damper 13 in the axial direction by the impact.

Here, the rubber damper 13 is in axial abutment against the two half members 3A and 3B of the anvil 3 through the protrusions 13c formed on the two faces of the damper members 13b, thereby to suppress the axial spring constant of the rubber damper 13 to a low value. As a result, the elastic deformation of the rubber damper 13 in the axial direction is enlarged to enhance the vibration absorptivity of the rubber damper 13 so that the axial vibrations are effectively absorbed by the rubber damper 13.

Thus, in this embodiment, the rubber damper 13 is interposed between the half member 3A and the half member 3B of the anvil 3 thereby to prevent the two half members 3A and 3B from directly contacting with each other in the rotational direction and in the axial direction. Even if relative torque occurs between the two half members 3A and 3B, the contact between the two half members 3A and 3B is prevented by the rubber damper 13 thereby to establish no frictional force in-between. Therefore, what obstructs the relative movements of the two half members 3A and 3B in the axial direction is only the reaction which is received from the rubber damper 13 by deforming the rubber damper 13 elastically, so that the axial shock absorptivity of the anvil 3 is enhanced. As a result, the axial vibrations to be propagated in the tip tool 4 are held low, so that the noise to be generated by the timber and occupying most of the noise in the timber screwing works are reduced to realize the noise reduction.

When the torque is applied to the anvil 3, on the other hand, the rubber damper 13 is elastically deformed so that the two half members 3A and 3B of the anvil 3 rotate relative to each other. A circumferential clearance is formed, while the torque is low, between the pawls 3c and 3f of the two half members 3A and 3B of the anvil 3. When the torque exceeds a predetermined value, the pawls 3c and the pawls 3f make direct contact (or metallic contact), as shown in FIG. 9(b), so that the torque is transmitted from the half member 3A to the half member 3B directly not through the rubber damper 13.

Now, when the pawls 3c and 3f of the half members 3A and 3B of the anvil 3 make the direct contact, as shown in FIG. 9(b), the sectional area (or the minimum sectional area) S1 of the space, which is defined by the recesses 3c-1 and 3f-1 of the pawls 3c and 3f, is set larger than the sectional area S2 of each of the damper members 13b of the rubber damper 13 (i.e., S1>S2), as shown in FIG. 8(a).

Here, the rubber damper 13 acts as the shock absorber in the rotational direction of the two half members 3A and 3B of the anvil 3. As a result, the impact sound, which is produced by the collision of the pawls 3c and 3f of the half members 3A and 3B, is reduced so that not only the sound emitted by the timber but also the noise emitted by the impact tool body is reduced.

From now on, similar actions are repeated to transmit the rotary impact intermittently and repeatedly from the tip tool 4 to a screw 11 so that the screw 11 is driven into the timber or the connection object.

Thus in the impact tool according to this embodiment, the transmission torque of the anvil 3 is increased to enlarge the relative rotations of the two half members 3A and 3B of the anvil 3 so that the space formed between the recesses 3c-1 and 3f-1 of the pawls 3c and 3f is reduced. However, the minimum sectional area S1 is set larger than the sectional area S2 of each of the damper members 13b of the rubber damper 13 arranged in that space (i.e., S1>S2), so that the elastic deformation of the rubber damper 13 is kept small so that the rubber damper 13 is prevented from being broken, thereby to improve its duration. Moreover, the loss of the impact energy by the elastic deformation of the rubber damper 13 (or the kinetic energy of the hammer 8) is reduced, so that a high fastening torque can be retained. As a result, the impact tool can also be applied to the works requiring the high torque such as the bolt fastening works so that its general versatility is enhanced.

In this embodiment, moreover, each of the damper members 13b of the rubber damper 13 is so arranged in the space formed between the recesses 3c-1 and 3f-1 of the pawls 3c and 3f of the anvil 3 that its longer axis is directed in the circumferential direction and that its shorter axis is directed in the radial direction. The longer axis length x of each of the damper members 13b is set equal to the enveloping circle diameter d of the space formed between the recesses 3c-1 and 3f-1 of the pawls 3c and 3f of the anvil 3 (i.e., x=d). The shorter axis length y of the same damper members 13b is set smaller than the enveloping diameter d (i.e., y<d). As a result, the sectional area S2 of the damper members 13b of the rubber damper 13 can be set smaller than the minimum sectional area S1 of the space which is formed between the recesses 3c-1 and 3f-1 of the pawls 3c and 3f of the anvil 3 (i.e., S2<S1), and the rubber damper 13 can be assembled without any looseness between the half members 3A and 3B of the anvil 3.

In this embodiment, moreover, the shorter axis length y (as referred to FIG. 8(a)) of each of the damper members 13b of the rubber damper 13 is set larger than the maximum value δ2max between the circumferential gap between the pawls 3c and 3f of the half members 3A and 3B of the anvil 3 (i.e., y>δ2max). Even if the damper member 13b should be disconnected by a cracking or the like from the connecting portion 13a, the damper member 13b disconnected is not flown away from the anvil 3 by a centrifugal force, but the shock absorbing action can be stably performed by the rubber damper 13.

In this embodiment, moreover, the rubber damper 13 is constituted by integrating the ring-shaped connecting portion 13a and the fourth damper members 13b, so that only one mold for molding the rubber damper 13 can be sufficed to reduce the production cost. Moreover, the shorter axis length y (as referred to FIG. 8(a)) of each of the damper members 13b is set larger than the maximum value δ2max (as referred to FIG. 9(b)) of the circumferential gap between the pawls 3c and 3f of the half members 3A and 3B of the anvil 3 (i.e., y>δ2max). Even if the damper member 13b should be disconnected by the cracking or the like from the connecting portion 13a, the damper member 13b disconnected is not flown away from the anvil 3 by a centrifugal force, but the shock absorbing action can be stably performed by the rubber damper 13.

In addition, the following effects can be attained according to this embodiment.

As shown in FIG. 5 and FIG. 7, more specifically, the confronting radially inner portions of the half members 3A and 3B of the anvil 3 have the sleeve-shaped protrusions 3g and 3h formed integrally in the axial directions, and the ring-shaped connecting portion 13a of the rubber damper 13 is fitted on the outer circumferences of those protrusions 3g and 3h. As a result, the rubber damper 13 and the spindle 7 are prevented from direct contact so that the rubber damper 13 is prevented from its wear thereby to improve its own durability. In this embodiment, the two half members 3A and 3B of the anvil 3 are provided with the axial protrusions 3g and 3h, respectively. If, however, one of these protrusions 3g and 3h is elongated, only one protrusion 3g or 3h can be formed at one half member 3A or 3B.

In this embodiment, moreover, the two axial end faces of each of the damper members 13b of the rubber damper 13 are positioned on the axially inner sides by Δx, as shown in FIG. 7, the end faces of the pawls 3c and 3f of the half members 3A and 3B of the anvil 3. As a result, the damper members 13b of the rubber damper 13 contact all over the axial width with the pawls 3c and 3f of the half members 3A and 3B of the anvil 3 so that the axial end faces of the pawls 3c and 3f of the half members 3A and 3B of the anvil 3 do not contact with the outer circumferences of the damper members 13b of the rubber damper 13. As a result, any high searing stress is not caused locally at the damper members 13b of the rubber damper 13 by the circumferentially relative rotations of the half members 3A and 3B of the anvil 3. Therefore, no cracking occurs in the damper members 13b of the rubber damper 13 so that the rubber damper 13 can be prevented from any damage thereby to improve its own durability. If the axial end faces of the pawls 3c and 3f of the half members 3A and 3B of the anvil 3 contact with the outer circumferences of the damper members 13b of the rubber damper 13, a high shearing stress occurs at the contacting portion (or the edge portion) thereby to cause the cracking in the damper members 13b of the rubber damper 13.

In this embodiment, moreover, the rubber damper 13 is brought into axial abutment against the half members 3A and 3B of the anvil 3 through the protrusions 13c protruded in the axial directions from the two faces of the individual damper members 13b, so that the spring constant of the rubber damper 13 in the axial direction is reduced. As a result, the elastic deformation of the rubber damper 13 in the axial direction is increased to enhance the vibration absorptivity of the rubber damper 13 so that the axial vibrations are effectively absorbed by the rubber damper 13 thereby to realize a more noise reduction.

Here, the rubber damper 13 to be used in the impact tool according to the invention may perform the shock absorbing function in both the axial direction and the rotational direction, and may act to prevent the direct contact between the two half members 3A and 3B of the anvil 3 during the actual operation in the axial direction and to cause the pawls 3c of the half member 3A of the anvil 3 to make direct contact with the pawls 3f of the half member 3B in the circumferential direction when a rotary torque at a set value or higher is applied. Thus, proper characteristics can be attained by changing the thickness of the rubber damper 13 and the angles of the pawls 3c and 3f of the half members 3A and 3B of the anvil 3 in accordance with the product specs. In case, moreover, there arises no problem on the product specs even if the transmission torque is set low, the angles of the pawls 3c and 3f of the two half members 3A and 3B of the anvil 3 are enlarged to keep the two half members 3A and 3B away from direct contact in the circumferential direction.

### INDUSTRIAL APPLICABILITY

The invention is useful especially for reducing the noise when applied to the impact tool such as a hammer drill for generating the rotary impact to perform the desired works.

What is claimed is:

1. An impact tool comprising:
a rotary impact mechanism mounted on a spindle to be rotationally driven by a motor,
wherein a rotary impact generated by said rotary impact mechanism is applied to a tip tool by transmitting the rotary impact intermittently from a hammer through an anvil to said tip tool;
a plurality of pawls which are formed on axially confronting faces of two half members formed by halving said anvil in an axial direction; and
a damper which is disposed in a space formed between said pawls arranged alternately in the circumferential direction of the two half members,
wherein the minimum sectional area S1 of said space formed between the pawls is set larger than the sectional area S2 of said damper.

2. An impact tool as set forth in claim 1, wherein said damper has a plurality of damper members of an elliptical column shape arranged in the circumferential direction around a ring-shaped connecting portion and formed integrally, and
wherein each of the damper members is arranged in the space formed between the pawls of the two half members of said anvil, so that its longer axis is directed in the circumferential direction whereas its shorter axis is arranged in the radial direction.

3. An impact tool as set forth in claim 2, wherein the longer axis length x of said damper members is set equal to the enveloping circle diameter d of the space formed between the pawls of the two half members of said anvil, and
wherein the shorter axis length y of said damper members is set smaller than the enveloping circle diameter d.

4. An impact tool as set forth in claim 2, wherein the shorter axis length y of said damper members is set larger than the maximum value δ2max of the inter-pawl gap between the two half members of said anvil.

5. An impact tool as set forth in claim 3, wherein the shorter axis length y of said damper members is set larger than the maximum value δ2max of the inter-pawl gap between the two half members of said anvil.

6. An impact tool as set forth in claim 1, wherein said damper is interposed between the two half members of the anvil in the axial direction, and
wherein vibration from the rotary impact mechanism or vibration source is absorbed to suppress the propagation of the vibration to the fastening object, thereby reducing noise of the impact tool.

7. An impact tool as set forth in claim 1, wherein transmission torque of the anvil is increased to enlarge relative rotations of the two half members of the anvil, wherein even if space formed between the pawls becomes small, a minimum section area S1 thereof is set larger than the section area S2 of the damper arranged in the space, and
wherein elastic deformation of the damper is reduced to prevent damage of the damper, thereby improving durability.

8. An impact tool as set forth in claim 2, wherein each of the damper members is so arranged in the space formed between the pawls of the two half member of the anvil that its longer axis is directed in the circumferential direction and its shorter axis if directed in the radial direction.

9. An impact tool as set forth in claim 3, wherein the longer axis length x of each of the damper members is set equal to an enveloping circle diameter d of the space formed between the pawls of the two half members of the anvil,
wherein the shorter axis length y of the same damper members is set smaller than the enveloping circule diameter d, and
wherein the sectional area S2 of the damper can be set smaller than the minimum sectional area S1 of the space between the pawls of the anvil, and the damper can be assembled without any looseness between the half members.

10. An impact tool as set forth in claim 4, wherein the shorter axis length y of the damper members is set larger than the maximum value δ2max of the inter-pawl gap between the two half members of the anvil, and
wherein if the damper member should be disconnected by a cracking from the connecting portion, the damper member disconnected is not flown away from the anvil by a centrifugal force, but the shock absorbing action by the damper can be stably performed.
